(19) United States
(12) Patent Application Publication
(10) Pub. No.: US 2016/0235286 A1
(43) Pub. Date: Aug. 18, 2016

(54) Medical Probe

(71) Applicant: EDA Medical Devices Technology Inc., Taichung City (TW)

(72) Inventors: Tai-An Chiang, Taichung City (TW); Tzeng-Ming Uen, Taichung City (TW)

(21) Appl. No.: 14/823,511

(22) Filed: Aug. 11, 2015

(30) Foreign Application Priority Data
Feb. 15, 2015 (TW) 104103805

(51) Int. Cl. A61B 1/313; A61B 1/015; A61B 1/04; A61B 1/00

(52) U.S. Cl. CPC A61B 1/313; A61B 1/00165; A61B 1/015; A61B 1/04

(57) ABSTRACT

A medical probe includes a hollow needle body, a light-transmitting optical module and an imaging module. The hollow needle body defines a needle passage extending along an axis of the needle body, and includes an outer tubular wall surrounding the needle passage. The outer tubular wall includes a penetrating section formed with a forward penetrating end, and a needle opening formed in the penetrating section and communicated with the needle passage. The light-transmitting optical module is disposed inside the needle passage, includes a light entering region and transmits the light within the needle passage. The imaging module is disposed in the needle passage to receive and process the light transmitted through the light-transmitting optical module.

### CROSS-REFERENCE TO RELATED APPLICATION

This application claims priority of Taiwanese Application No. 104103805, filed on Feb. 4, 2015.

### FIELD

The disclosure relates to a medical probe, and more particularly to a medical probe adapted for examining a body cavity of a patient during surgery.

### BACKGROUND

A conventional medical probe device for penetrating and examining a body cavity of a patient is generally applied to a minimally invasive surgery (e.g., a front artificial pneumoperitoneum) in order to improve accuracy of a penetrating process during the surgery.

Referring to FIG. 1, the medical probe device 1, as disclosed in Taiwanese Patent No. 250437, includes a probe body 11 and an optical fiber 12. The probe body 11 has an end part 111 that is formed at a front end of the probe body 11 and a concave part 112 that is formed at one side of the probe body 11. The optical fiber 12 has a viewing tip 121 at a front end of the optical fiber 12. The viewing tip 121 of the optical fiber 12 is may be disposed at the end part 111, or in the concave part 112 of the probe body 11 such that the viewing tip 121 may capture images in front of the probe body 11 during the penetrating process of the surgery.

However, if the viewing tip 121 is disposed in the concave part 112 of the probe body 11, because the viewing angle of the viewing tip 121 may be limited due to blockage by the end part 111 of the probe body 11, it will be difficult to verify an image incident on the viewing tip 121.

If the viewing tip 121 is disposed at the end part 111 of the probe body 11 in order to obtain a wider viewing angle, the end part 111 requires an additional part for supporting the viewing tip 121, which can increase a cross-section of the end part 111 and thus reduce a penetrating effect of the probe body 11.

### SUMMARY

Therefore, an object of the disclosure is to provide a medical probe that can alleviate at least one of the drawbacks of the prior arts.

According to the disclosure, a medical probe is for examining a body cavity during surgery. The medical probe includes a hollow needle body, a light-transmitting optical module and an imaging module.

The hollow needle body defines a needle passage that extends along an axis of the hollow needle body, and includes an outer tubular wall that surrounds the needle passage. The outer tubular wall includes a penetrating section that is formed with a forward penetrating end and that has a cross-section smaller than a remainder of the outer tubular wall, and a needle opening that is formed in the penetrating section and that is communicated with the needle passage.

The light-transmitting optical module is disposed inside the needle passage, and includes a light entering region that extends to and exposed from the needle opening to receive light from the body cavity. The light-transmitting optical module transmits the light within the needle passage.

The imaging module is disposed in the needle passage to capture an image carried by the light transmitted through the light-transmitting optical module.

### BRIEF DESCRIPTION OF THE DRAWINGS

Other features and advantages of the disclosure will become apparent in the following detailed description of the embodiments with reference to the accompanying drawings, of which:

FIG. 1 is a fragmentary cross-sectional view of a medical probe device disclosed in Taiwanese Patent No. 250437.

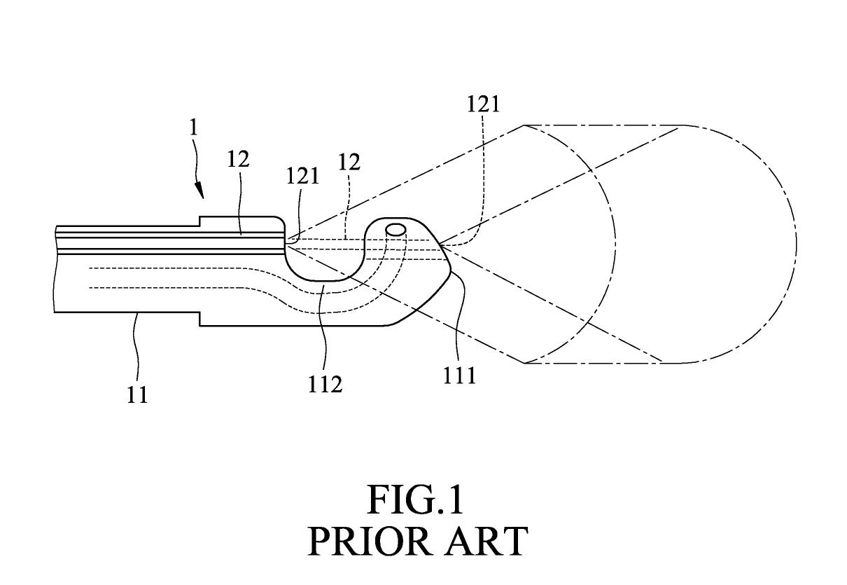

FIG. 2 is a fragmentary exploded perspective view of a first embodiment of a medical probe according to the present disclosure;

FIG. 3 is a fragmentary perspective view of the first embodiment according to the present disclosure;

FIG. 4 is a fragmentary sectional view of the first embodiment according to the present disclosure;

FIG. 5 is a cross-sectional view of the medical probe taken along line V-V in FIG. 4;

FIG. 6 is a fragmentary schematic view of a second embodiment of the medical probe according to the present disclosure; and

FIG. 7 is a fragmentary sectional view of a third embodiment of the medical probe according to the present disclosure.

### DETAILED DESCRIPTION

Before the disclosure is described in greater detail, it should be noted that like elements are denoted by the same reference numerals throughout the disclosure.

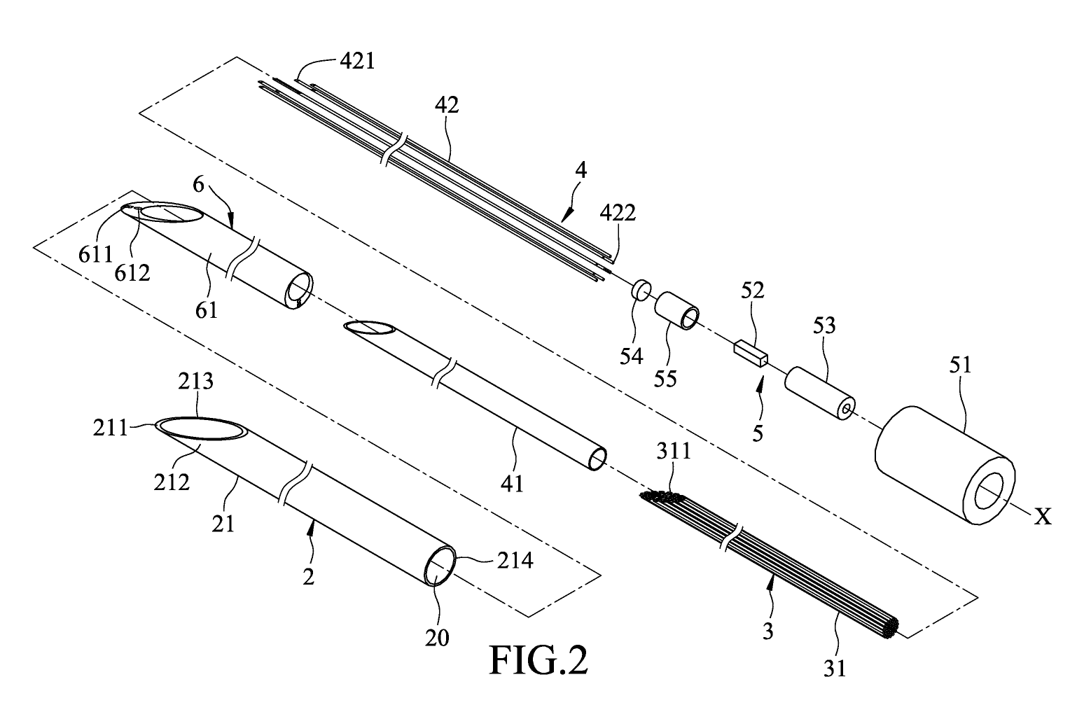

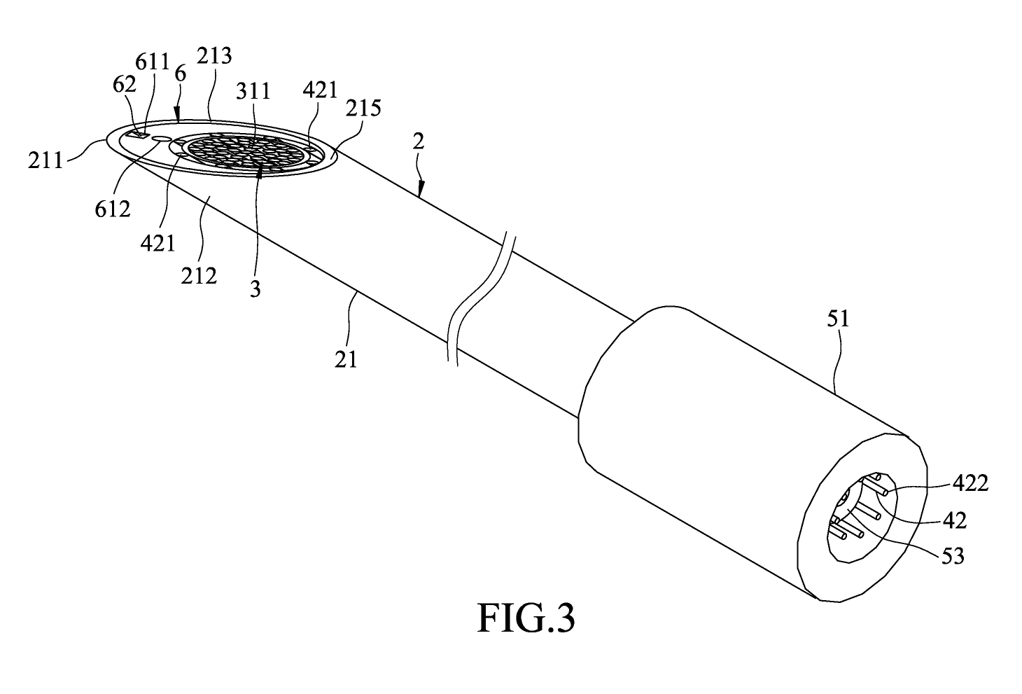

Referring to FIGS. 2 to 5, a first embodiment of a medical probe according to the present disclosure is used for examining a body cavity of a patient. The medical probe includes a hollow needle body 2, a light-transmitting optical module 3, a light-emitting module 4, an imaging module 5 and a microelectro-mechanical module 6.

The hollow needle body 2 defines a needle passage 20 extending along an axis (X) of the hollow needle body 2, and includes an outer tubular wall 21 surrounding the needle passage 20. The outer tubular wall 21 includes a penetrating section 212 that is formed with a forward penetrating end 211 and that has a cross-section smaller than a remainder of the outer tubular wall 21, and a rearward end 214 opposite to the forward penetrating end 211. The outer tubular wall 21 is formed, in the penetrating section 212 thereof, with a needle opening 213 that is communicated with the needle passage 20. In this embodiment, the outer tubular wall 21 is beveled to form the penetrating section 212 so that the penetrating section 212 has a beveled annular surface 215 that is inclined with respect to the axis (X) of the hollow needle body 2 and that confines the needle opening 213. The forward penetrating end 211 of the penetrating section 212 of the outer tubular wall 21 is situated at one side of the beveled annular surface 215 offset from the axis (X) of the hollow needle body 2. The hollow needle body 2 further includes an adapter 51 that is connected to the rearward end 214 of the outer tubular wall 21. The needle passage 20 of the needle body 2 is surrounded by the outer tubular wall 21 and the adapter 51.

The light-transmitting optical module 3 is disposed inside the needle passage 20 of the hollow needle body 2, and includes a plurality of optical fibers 31. The optical fibers 31 are disposed inside the needle passage 20 of the hollow needle body 2 along the axis (X) between the forward penetrating end 211 and the imaging module 5. The optical fibers 31 are beveled to respectively have beveled surfaces that cooperatively form a light entering region 311 in the needle opening 213 to receive light from the body cavity. The light-transmitting optical module 3 transmits the light within the needle passage 20.

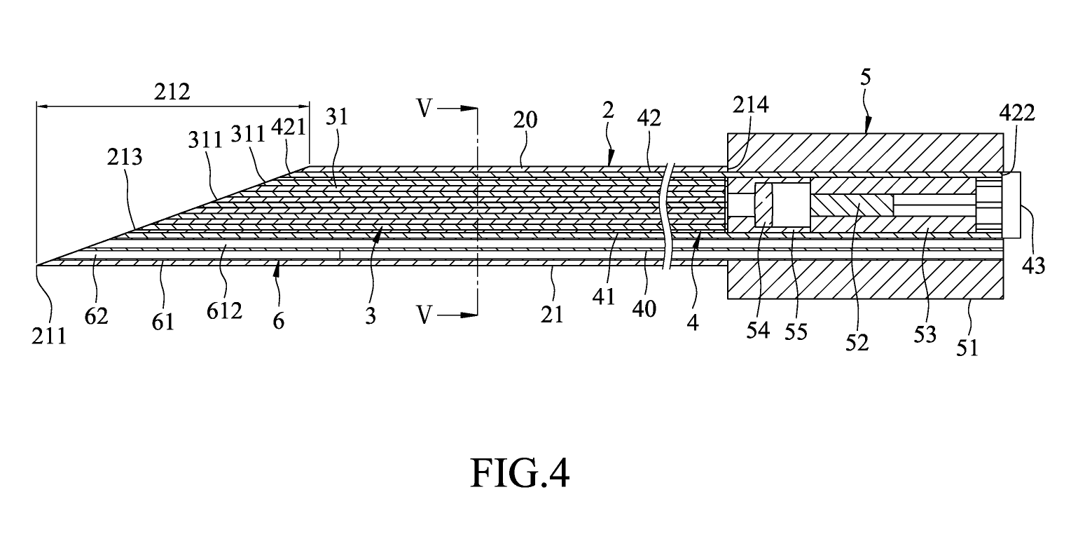

The light-emitting module 4 is to emit light to pass through the needle passage 20 and to deliver the light to the body cavity through the needle opening 213. The hollow needle body 2 further includes an inner tubular wall 41 that is disposed within the outer tubular wall 21 along the axis (X) of the hollow needle body 2 and that surrounds the optical fibers 31 of the light-transmitting optical module 3. The light-emitting module 4 includes a plurality of light guide rods 42 that extend along the axis (X) of the hollow needle body 2 and that are disposed around the inner tubular wall 41, and a light emitter 43 that is distal from the needle opening 213 (particularly, at a rearward end of the adapter 51 as shown in FIG. 4). Each of the light guide rods 42 has a light entrance end 422 that is proximal to the light emitter 43 to receive light emitted from the light emitter 43, and alight exit end 421 that extends to and is exposed from the needle opening 213 of the hollow needle body 2 to deliver the light through the needle opening 213 to the body cavity. The inner tubular wall 41 cooperates with the outer tubular wall 21 to define a passageway 40 that extends along the axis (X) of the hollow needle body 2. The light emitter 43 may be one of a light-emitting diode (LED), laser, cool light, etc.

The imaging module 5 is disposed in the needle passage 20 (particularly, in a portion of the needle passage 20 surrounded by the adapter 51) to capture an image carried by the light transmitted through the light-transmitting optical module 3. The imaging module 5 includes an image sensor 52 that is disposed inside the adapter 51, a sensor carrier 53 that is inserted into the adapter 51 and that carries the image sensor 52, a lens 54 that is disposed between the light-transmitting optical module 3 and the image sensor 52 to focus the light, and a lens carrier 55 that is inserted into the adapter 51 and that carries the lens 54.

The microelectro-mechanical module 6 includes a support body 61 that surrounds the axis (X) of the hollow needle body 2 and that is disposed inside the needle passage 20 of the hollow needle body 2 from the needle opening 213 to the rearward end of the adapter 51, and a microelectro-mechanical component 62 that is disposed in the support body 61 proximally of the needle opening 213 to detect physiological characteristics of the body cavity. The support body 61 has a mounting hole 611, and a channel hole 612 that is formed in the support body for guiding a fluid flow. The mounting hole 611 and the channel hole 612 penetrate through the support body 61 in the direction parallel to the axis (X) of the outer tubular wall 21 and extend to the needle opening 213. In clinical implementation, the mounting hole 611 may receive the microelectro-mechanical component 62 and permit passage of electrical cables (not shown)

While the light emitter 43 is disposed inside the adapter 51 in the embodiment, it may also be disposed outside the adapter 51 in other embodiments of the present disclosure. On the other hand, the image sensor 52 and the microelectro-mechanical component 6 may be connected electrically to a control device (not shown) such that the control device is able to verify information received from the image sensor 52 and the microelectro-mechanical component 6.

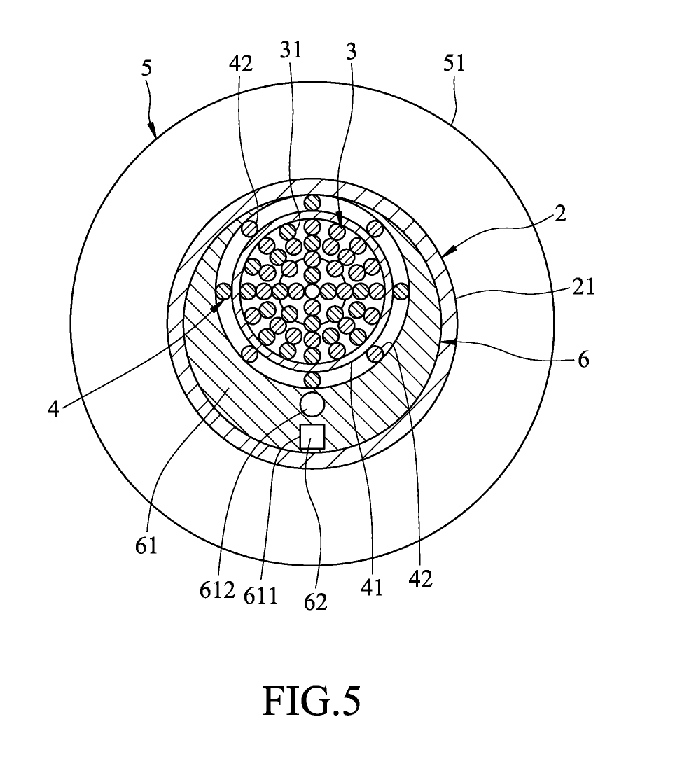

Referring to FIGS. 4 and 5, by virtue of the forward penetrating end 211 and the penetrating section 212 of the hollow needle body 2, the medical probe may penetrate into the body cavity of a patient during the penetrating process of a surgery. Meanwhile, the light emitter 43 emits the light to propagate from the light entrance ends 422 of the light guide rods 42 to the light exit ends 421 of the light guide rods 42 such that the light is delivered through the needle opening 213 to the body cavity in front of the forward penetrating end 211 of the hollow needle body 2.

Since the light entering region 311 of the optical fibers 31 is small and face forwardly from the needle opening 213 of the hollow needle body 2, the light reflected from the body cavity to the needle body 2 may enter the light entering region 311 and propagate through the optical fibers 31 inside the needle passage 20, and may be focused by the lens 54 for detection by the image sensor 52 so as to obtain image in front of the hollow needle body 2. If an abnormal condition (e.g., tissue adhesion) is observed during the penetrating process of the surgery, the location to be penetrated may be changed to avoid high-risk area.

It is worth mentioning that a syringe (not shown) maybe connected to the channel hole 612 via the adapter 51 so as to inject liquors and/or testing liquid from the syringe to the body cavity through the channel hole 612, or to draw body fluid from the cavity to the syringe through the channel hole 612. In addition to that, the microelectro-mechanical component 62 disposed in the mounting hole 611 may detect physiological characteristics according to the body fluid and the tissue inside the cavity.

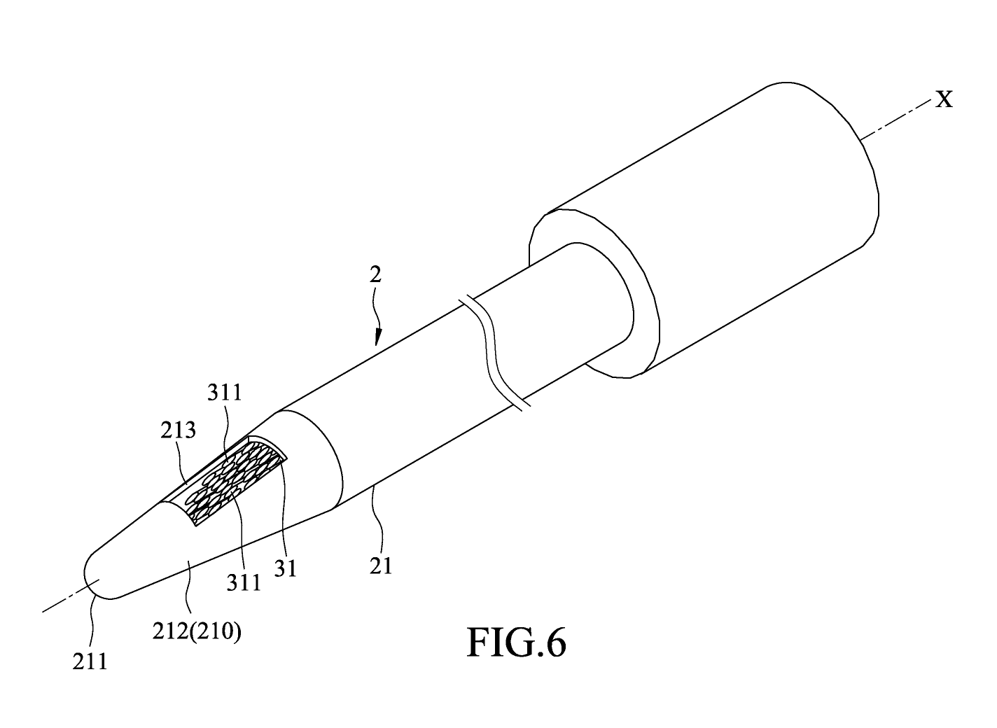

Referring to FIG. 6, a second embodiment of the medical probe according to the present disclosure is shown to be similar to the first embodiment. The only difference resides in that the forward penetrating end 211 is blunt and situated on the axis (X) of the hollow needle body 2. The outer tubular wall 21 converges in the penetrating section 212 to form a converging wall section 210 that constitutes the penetrating section 212. The needle opening 213 is formed in one side of the converging wall section 210 to expose the light entering region 311 of the light-transmitting optical module 3.

Even though the blunt forward penetrating end 211 in this embodiment cannot directly penetrate skin and muscle of the patient, the medical probe of this embodiment may enter the body cavity of the patient after the skin and muscle is cut open by a surgical knife (e.g., a scalpel). In this embodiment, the effectiveness of the first embodiment may still be achieved.

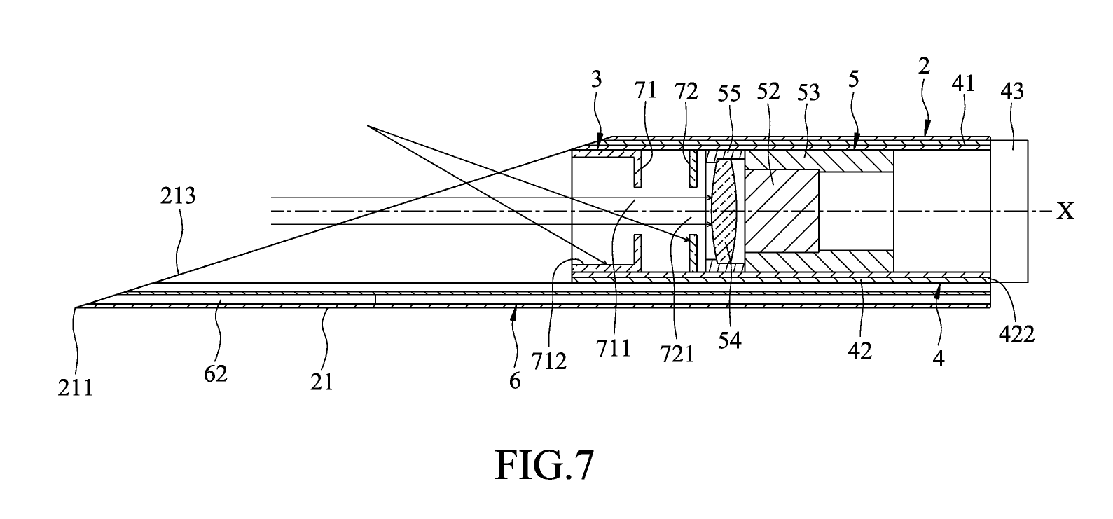

Referring to FIG. 7, a third embodiment of the medical probe according to the present disclosure is shown, and is generally similar to the first embodiment. However, the needle passage 20 of the needle body 2 is in this embodiment is surrounded only by the outer tubular wall 21. The light emitter 43 is disposed at the rearward end 214 of the outer tubular wall 21, the light entrance ends 422 of the light guide rods 42 extend to the light emitter 43. The light-transmitting optical module 3 further includes first and second optical gratings 71, 72 that are disposed in the needle passage 20 in proximity to the needle opening 213. The first and second optical gratings 71, 72 have respectively first and second grating holes 711, 721 that are used as the light entering region 311. The axis (X) of the hollow needle body 2 extends through the first and second grating holes 711, 721. The first optical grating 71 in this embodiment further includes an inner surface 712 that surrounds the axis (X) of the hollow needle body 2 and that forms a light-unreflecting coating thereon.

The imaging module 5 in this embodiment is disposed in the needle passage 20 and is disposed within the inner tubular wall 41 of the needle body 2. The imaging module 5 includes an image sensor 52, a sensor carrier 53 carrying the image sensor 52, a lens 54 disposed between the second optical grating 72 and the image sensor 52, and a lens carrier 55 carrying the lens 54. The lens 54 is to focus the light reflected from the body cavity and passing through the first and second grating holes 711, 721.

The first and second grating holes 711, 721 only permit passage of the reflected light from the body cavity in front of the hollow needle body 2. Therefore, the reflected light coming from an upside of the hollow needle body 2 is excluded while the remaining reflected light passes through the first and second grating holes 711, 721. The reflected light passing through the first and second grating holes 711, 721 will propagate within the needle passage 20 and will be focused by the lens 54 so that an image in front of the hollow needle body 2 may be captured by the image sensor 52. In this embodiment, the effects of the first embodiment may still be achieved.

To conclude, the medical probe according to the present disclosure has the following advantages and effectiveness:

By virtue of the unique location designed for the light entering region 311 of the light-transmitting optical module 3, the imaging module 5 is able to capture the images of the body cavity in front of the hollow needle body 2 when the hollow needle body 2 penetrates the body cavity, thereby improving accuracy in determination of a proper surgical site for a surgery and reducing risks during the surgery. While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A medical probe for examining a body cavity during surgery, the medical probe comprising:
a hollow needle body defining a needle passage extending along an axis of said hollow needle body, and including an outer tubular wall surrounding said needle passage, said outer tubular wall including a penetrating section that is formed with a forward penetrating end and that has a cross-section smaller than a remainder of said outer tubular wall, and a needle opening formed in said penetrating section and communicated with said needle passage;
a light-transmitting optical module disposed inside said needle passage, and including a light entering region that extends to and is exposed from said needle opening to receive light from the body cavity, said light-transmitting optical module transmitting the light within said needle passage; and
an imaging module disposed in said needle passage to capture an image carried by the light transmitted through said light-transmitting optical module.

2. The medical probe of claim 1, wherein said outer tubular wall is beveled to form said penetrating section so that said penetrating section has a beveled annular surface that is inclined with respect to the axis of said hollow needle body and that confines said needle opening, said forward penetrating end being situated at one side of said beveled annular surface offset from the axis.

3. The medical probe of claim 1, wherein said forward penetrating end is blunt and is situated on the axis of said hollow needle body, said outer tubular wall converging in said penetrating section to form a converging wall section that constitutes said penetrating section, said needle opening being formed in one side of said converging wall section.

4. The medical probe of claim 1, further comprising a microelectro-mechanical module, which includes a support body disposed inside said needle passage, and a microelectro-mechanical component disposed in said support body proximally of said needle opening to detect physiological characteristics of the body cavity.

5. The medical probe of claim 4, wherein said support body has a channel hole formed therein for guiding a fluid flow.

6. The medical probe of claim 5, wherein said channel hole penetrates through said support body in a direction parallel to the axis of said outer tubular wall, said support body further having a mounting hole to receive said microelectro-mechanical component.

7. The medical probe of claim 1, wherein said light-transmitting optical module further includes a plurality of optical fibers that are disposed inside said needle passage along the axis and that extend from said needle opening to said imaging module, said optical fibers being beveled to respectively have beveled surfaces that cooperatively form said light entering region of said light transmitting optical module in said needle opening.

8. The medical probe of claim 1, wherein said light-transmitting optical module further includes an optical grating that is disposed in said needle passage in proximity to said needle opening and that has a grating hole to be used as said light entering region, the axis of said hollow needle body extending through said grating hole.

9. The medical probe of claim 1, further comprising a light-emitting module to emit light to pass through said needle passage and to deliver the light to the body cavity through said needle opening.

10. The medical probe of claim 9, wherein said hollow needle body further includes an inner tubular wall disposed within said outer tubular wall and surrounding said light-transmitting optical module, said light-emitting module including a plurality of light guide rods extending along the axis of said hollow needle body and disposed around said inner tubular wall, and a light emitter distal from said needle opening, each of said light guide rods having a light entrance end proximal to said light emitter to receive light from said light emitter, and a light exit end extending to and exposed from said needle opening to deliver the light through said needle opening to the body cavity.

11. The medical probe of claim 10, wherein said imaging module includes an image sensor, a sensor carrier carrying said image sensor and inserted into said inner tubular wall, a lens disposed between said light-transmitting optical module and said image sensor to focus the light, and a lens carrier carrying said lens and inserted into said inner tubular wall.

12. The medical probe of claim 10, wherein said hollow needle body further includes an adapter connected to a rearward end of said outer tubular wall oppositely of said forward penetrating end, said imaging module including an image sensor disposed inside said adapter, a sensor carrier inserted in said adapter and carrying said image sensor, a lens disposed between said light-transmitting optical module and said image sensor to focus light, and a lens carrier inserted into said adapter and carrying said lens.
